SEQUENCE LISTING (2 sequences)

SEQ ID NO: 1 - DNA, 601 bp, Homo sapiens
gcgccagctggcttgcccgagaccccggcccaatctacagactgggaaactgaggctagccctgcggcgctcacctcagcagggcaggcaggaggcgccggcagcctctggccctcctccagcagttccaagaggcggcagagggcggggacatcccgctcacatcccatcatccgcaggaactcctggagccaaggaggagcgcatgtggtgggggaggagcctccgtgggtggagggggaggggccacccgtggtgggggtaggggccatctgtgatggagcggggaggggctgcctgmaccgtgaaggggtcagaaaggtgaggaagcgtcctccccactggggcctatgatgctgggaccaggtgaccccatgctaaagagggacgcaggcgcagacaggttggagattggccacgaggggcgtggagggagaagaaggctgggggctctgggaagccgactcacggccgaggggctgcagcttttgtcgcagtaggtgaagagctcgtacaggacgaccccgaagctccagacgtctgactggcgagagaagatgttgtccgagagggattcgggggcatacctggagaggggaca

SEQ ID NO: 2 - DNA, 511 bp, Homo sapiens
gcccggctattttttttgtattttcagtagcgacggggtttcacggtgttagccaggatggtgttgatctcctgacctcgtgatccacctgcctcggcctcccaaaatgctgggattacaggcgtgagccacggcgcctggcccatggctgattttataaatggggggagggtgtcacctggcaaggatcccagggctacagaggtacctgaatttgagcccaggtctctctgtcttcttctatctctgactcctycccattccctctcaccttcccccacagtctggactttgccatcaacaagctcaagactgggggctcacgtcctggctcctatgttctccgccgcagcccccaggactttgacagcttcctcctcactgtctgtgtccaggtcggtctactgctagggtgggtagtggagggctgcctggaggaggtgacgtttgaattgagatttaaaagatcagtcagcatttggttcctgaagaataggagggaaaagacacc

(19) United States
(12) Patent Application Publication
(10) Pub. No.: US 2011/0189685 A1
(43) Pub. Date: Aug. 4, 2011

(54) METHODS OF USING JAK3 GENETIC VARIANTS TO DIAGNOSE AND PREDICT CROHN'S DISEASE

(75) Inventors: Kent D. Taylor, Ventura, CA (US); Jerome I. Rotter, Los Angeles, CA (US); Ling Mei, Pasadena, CA (US); Stephan R. Targan, Santa Monica, CA (US)

(73) Assignee: CEDARS-SINAI MEDICAL CENTER, Los Angeles, CA (US)

(21) Appl. No.: 13/124,311

(22) PCT Filed: Oct. 22, 2009
(86) PCT No.: PCT/US09/61698

§ 371 Date: Apr. 14, 2011

Related U.S. Application Data
(60) Provisional application No. 61/107,590, filed on Oct. 22, 2008.

(51) Int. Cl. C12Q 1/68

(52) U.S. Cl. 435/6.12

(57) ABSTRACT

The present invention relates to methods of diagnosing and diagnosing susceptibility to Crohn's Disease by determining the presence or absence of risk variants at the JAK3 locus. In one embodiment, the present invention provides a method of diagnosing susceptibility to Crohn's Disease by determining the presence of a risk variant at the JAK3 locus, where the risk variant is associated with positive expression of ASCA and/or anti-I2.

### GOVERNMENT RIGHTS

This invention was made with U.S. Government support on behalf of the National Institute of Diabetes and Digestive and Kidney Diseases (NIDDK) by NIDDK Grant P01DK046763. The U.S. Government may have certain rights in this invention.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

### SUMMARY OF THE INVENTION

Various embodiments include a method of diagnosing susceptibility to a subtype of Crohn's disease in an individual, comprising determining the presence or absence of one or more risk variants at the Janus kinases 3 (JAK3) genetic locus in the individual, and determining the presence or absence of a positive expression of ASCA and/or anti-I2, where the presence of one or more risk variants at the JAK3 locus and the presence of ASCA and/or anti-I2 expression is indicative of susceptibility in the individual to the subtype of Crohn's Disease. In another embodiment, one of the one or more risk variants at the JAK3 locus comprises SEQ. ID. NO.: 1. In another embodiment, one of the one or more risk variants at the JAK3 locus comprises SEQ. ID. NO.: 2. In another embodiment, positive expression of ASCA and/or anti-I2 comprises a high level of expression relative to a healthy subject.

Other embodiments include a method of diagnosing a subtype of Crohn's disease in an individual, comprising obtaining a sample from the individual, assaying the sample for the presence or absence of a risk variant at the Janus kinases 3 (JAK3) genetic locus in the individual, and diagnosing the subtype of Crohn's disease based upon the presence of the risk variant at the JAK3 genetic locus. In another embodiment, the risk variant comprises SEQ. ID. NO.: 1 and/or SEQ. ID. NO.: 2. In another embodiment, the presence of the risk variant is associated with a positive expression of ASCA and/or anti-I2. In another embodiment, the positive expression of ASCA and/or anti-I2 comprises a high level of expression relative to a healthy subject.

Other features and advantages of the invention will become apparent from the following detailed description, taken in conjunction with the accompanying drawings, which illustrate, by way of example, various embodiments of the invention.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed, J. Wiley & Sons (New York, N.Y. 2001); and Sambrook and Russel, Molecular Cloning: A Laboratory Manual 3rd ed, Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Haplotype” as used herein refers to a set of single nucleotide polymorphisms (SNPs) on a gene or chromatid that are statistically associated.

“Risk” as used herein refers to an increase in susceptibility to IBD, including but not limited to CD and UC.

“Protective” and “protection” as used herein refer to a decrease in susceptibility to IBD, including but not limited to CD and UC.

“CD” and “UC” as used herein refer to Crohn's Disease and Ulcerative colitis, respectively.

“Jak3” as used herein refers to Janus kinase 3.

As used herein, examples of SNP variants rs2302600 and rs3212741 at the Jak3 genetic locus are described herein as SEQ. ID. NO.: 1 and SEQ. ID. NO.: 2, respectively. However, as understood by one of skill in the art, additional risk variants the Jak2 genetic locus may be readily apparent to one of skill in the art and Jak3 risk variants are not limited to these specific SNP sequences. Similarly, SNP variants rs2302600 and rs3212741 themselves may also come in many additional versions, including for example, nucleotide probes encoding the complementary strands.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

The inventors performed a genome-wide association study testing autosomal single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to CD. These SNPs and haplotypes are suitable for genetic testing to identify at risk individuals and those with increased risk for complications associated with serum expression of Anti-Saccharomyces cerevisiae antibody, and antibodies to I2, OmpC, and Cbir. The detection of protective and risk SNPs and/or haplotypes may be used to identify at risk individuals predict disease course and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease and ulcerative colitis. Other embodiments provide for methods of prognosing inflammatory bowel disease including but not limited to Crohn's Disease and ulcerative colitis. Other embodiments provide for methods of treating inflammatory bowel disease including but not limited to Crohn's Disease and ulcerative colitis.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease and ulcerative colitis, as described herein. The methods may also further include recording whether a genetic risk, susceptibility for inflammatory bowel disease including but not limited to Crohn's Disease and ulcerative colitis exists in the individual. The methods may also further include a prognosis of inflammatory bowel disease based upon the presence or absence of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

As disclosed herein, the inventors investigated the role genetic variants in the gene JAK3 may have in the development of Crohn's Disease. The inventors performed an antibody genome wide association study using patients diagnosed with Crohn's Disease, and found an association of JAK3 variants with expression of anti-I2 and ASCA for Crohn's Disease. The results of these studies are described in Tables 1-19 herein.

In one embodiment, the present invention provides a method of diagnosing susceptibility to a subtype of Crohn's Disease by determining the presence or absence of a risk variant at the JAK3 locus, where the presence of the risk variant at the JAK3 locus is indicative of susceptibility to the subtype of Crohn's Disease. In another embodiment, the risk variant is associated with ASCA and/or anti-I2 expression. In another embodiment, the risk variant at the JAK3 locus comprises SEQ. ID. NO.: 1. In another embodiment, the risk variant at the JAK3 locus comprises SEQ. ID. NO.: 2.

In one embodiment, the present invention provides a method of diagnosing Crohn's Disease by determining the presence or absence of a risk variant at the JAK3 locus, where the presence of the risk variant at the JAK3 locus is indicative of Crohn's Disease. In another embodiment, the risk variant is associated with ASCA and/or anti-I2 expression. In another embodiment, the risk variant at the JAK3 locus comprises SEQ. ID. NO.: 1. In another embodiment, the risk variant at the JAK3 locus comprises SEQ. ID. NO.: 2.

In another embodiment, the present invention provides a method of treating Crohn's Disease by determining the presence of a risk variant at the JAK3 locus and treating the Crohn's Disease.

In one embodiment, the present invention provides a method of determining protection against inflammatory bowel disease in an individual by determining the presence or absence of a protective haplotype at the JAK3 locus, where the presence of a protective haplotype at the JAK3 locus is indicative of a decreased likelihood of inflammatory bowel disease.

There are many techniques readily available in the field for detecting the presence or absence of antibodies, polypeptides or other biomarkers, including protein microarrays. For example, some of the detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Similarly, there are any number of techniques that may be employed to isolate and/or fractionate antibodies or protein biomarkers. For example, a biomarker and/or antibody may be captured using biospecific capture reagents, such as aptamers or other antibodies that recognize the antibody and/or protein biomarker and modified forms of it. This method could also result in the capture of protein interactors that are bound to the proteins or that are otherwise recognized by antibodies and that, themselves, can be biomarkers. The biospecific capture reagents may also be bound to a solid phase. Then, the captured proteins can be detected by SELDI mass spectrometry or by eluting the proteins from the capture reagent and detecting the eluted proteins by traditional MALDI or by SELDI. One example of SELDI is called “affinity capture mass spectrometry,” or “Surface-Enhanced Affinity Capture” or “SEAC,” which involves the use of probes that have a material on the probe surface that captures analytes through a non-covalent affinity interaction (adsorption) between the material and the analyte. Some examples of mass spectrometers are time-of-flight, magnetic sector, quadrupole filter, ion trap, ion cyclotron resonance, electrostatic sector analyzer and hybrids of these.

Alternatively, for example, the presence of biomarkers such as polypeptides and antibodies may be detected using traditional immunoassay techniques. Immunoassay requires biospecific capture reagents, such as antibodies, to capture the analytes. The assay may also be designed to specifically distinguish protein and modified forms of protein, which can be done by employing a sandwich assay in which one antibody captures more than one form and second, distinctly labeled antibodies, specifically bind, and provide distinct detection of, the various forms. Antibodies can be produced by immunizing animals with the biomolecules. Traditional immunoassays may also include sandwich immunoassays including ELISA or fluorescence-based immunoassays, as well as other enzyme immunoassays.

Prior to detection, antibodies and/or biomarkers may also be fractionated to isolate them from other components in a solution or of blood that may interfere with detection. Fractionation may include platelet isolation from other blood components, sub-cellular fractionation of platelet components and/or fractionation of the desired biomarkers from other biomolecules found in platelets using techniques such as chromatography, affinity purification, 1D and 2D mapping, and other methodologies for purification known to those of skill in the art. In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

Similarly, a variety of methods can also be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### JAK3 Variant (rs2302600) Associated with Anti-I2 Expression (Positive/Negative)

### Table 1

TABLE 1
Results demonstrating the association of anti-I2 as positive/negative
expression with JAK3 SNP rs2302600 (SEQ. ID. NO.: 1) as a result
of GWAS. Mantel-Haenszel Chi-Square statistics for the degree
of freedom (DF), value and probability of anti- I2 antibody expression
associated with genotype alleles AA, CA and CC for SEQ. ID.
NO.: 1 at the JAK3 genetic locus.

| | rs2302600 | | |
|---|---|---|---|
| I2_P(I2_P) | AA | CA | CC |
| Positive | 76 | 64 | 19 |
| | 47.8 | 40.25 | 11.95 |
| negative | 54 | 24 | 7 |
| | 63.53 | 28.24 | 8.24 |
| Statistic | DF | Value | Prob |
| Mantel-Haenszel | 1 | 4.5573 | 0.0328 |
| Chi-Square | | | |

### Example 2

### JAK3 Variant (rs2302600) Associated with Anti-I2 Expression Under Dominant Genetic Model

### Table 2

TABLE 2
Results demonstrating the association of anti-I2 with JAK3 SNP
rs2302600 (SEQ. ID. NO.: 1) under dominant genetic model.

| | rs2302600_dom | | |
|---|---|---|---|
| I2_P(I2_P) | 0 | 1 | Total |
| Positive | 76 | 83 | 159 |
| | 47.8 | 52.2 | |
| negative | 54 | 31 | 85 |
| | 63.53 | 36.47 | |
| Statistic | DF | Value | Prob |
| Chi-Square | 1 | 5.5062 | 0.0189 |

### Example 3

### JAK3 Variant (rs2302600) Associated with ASCA Expression Under Dominant Genetic Model

### Table 3

TABLE 3
Results demonstrating the association of ASCA with JAK3 SNP
rs2302600 (SEQ. ID. NO.: 1) under dominant genetic model.

| | rs2302600_dom | | |
|---|---|---|---|
| ASCA | 0 | 1 | Total |
| Positive | 76 | 80 | 156 |
| | 48.72 | 51.28 | |
| negative | 55 | 36 | 91 |
| | 60.44 | 39.56 | |
| Statistic | DF | Value | Prob |
| Chi-Square | 1 | 3.1704 | 0.075 |

### Example 4

### JAK3 Variant (rs2302600) Associated with Anti-I2 Level

### Table 4

TABLE 4
Results demonstrating the association of JAK3 variant rs2302600 (SEQ.
ID. NO.: 1) with anti-12 level in Crohn's Disease patients.
Analysis Variable: I2VALUE I2 VALUE

| | N | | |
|---|---|---|---|
| rs2302600_dom | Obs | N | Median |
| 0 | 132 | 130 | 26.745 |
| 1 | 116 | 114 | 37.559 |

P = 0.03

### Example 5

### JAK3 Variant (Rs2302600) Associated with ASCA Level

### Table 5

TABLE 5
Results demonstrating the association of JAK3 variant rs2302600 (SEQ.
ID. NO.: 1) with ASCA level in Crohn's Disease patients
Analysis Variable: ascalev

| | N | | |
|---|---|---|---|
| rs2302600_dom | Obs | N | Median |
| 0 | 132 | 131 | 0.3021 |
| 1 | 116 | 116 | 0.6011 |

P = 0.02

### Example 6

### JAK3 Variant (rs3212741) Associated with ASCA Expression (Positive/Negative)

### Table 6

TABLE 6
Results demonstrating the association of ASCA as positive/negative
expression with JAK3 SNP rs3212741 (SEQ. ID. NO.: 2) as a
result of GWAS. Mantel-Haenszel Chi-Square statistics for
the degree of freedom (DF), value and probability of ASCA
antibody expression associated with genotype alleles CC, TC,
and TT for SEQ. ID. NO.: 2 at the JAK3 genetic locus.

| | rs3212741 | | |
|---|---|---|---|
| ASCA | CC | TC | TT |
| Positive | 113 | 40 | 2 |
| | 72.9 | 25.81 | 1.29 |
| negative | 54 | 34 | 2 |
| | 60 | 37.78 | 2.22 |
| Statistic | DF | Value | Prob |
| Mantel-Haenszel | 1 | 4.2511 | 0.0392 |
| Chi-Square | | | |

### Example 7

### JAK3 Variant (rs3212741) Associated with ASCA Expression Under Dominant Genetic Model

### Table 7

TABLE 7
Results demonstrating the association of JAK3 SNP rs3212741
(SEQ. ID. NO.: 2) under dominant genetic model.

| | rs3212741_dom | | |
|---|---|---|---|
| ASCA | 0 | 1 | Total |
| Positive | 113 | 42 | 155 |
| | 72.9 | 27.1 | |
| negative | 54 | 36 | 90 |
| | 60 | 40 | |
| Statistic | DF | Value | Prob |
| Chi-Square | 1 | 4.3684 | 0.0366 |

### Example 8

### JAK3 Variant (rs3212741) Associated with ASCA Level

### Table 8

TABLE 8
Results demonstrating the association of JAK3 variant rs3212741 (SEQ.
ID. NO.: 2) with ASCA level in Crohn's Disease patients.
Analysis Variable: ascalev

| | N | | |
|---|---|---|---|
| rs3212741_dom | Obs | N | Median |
| 0 | 167 | 167 | 0.561 |
| 1 | 79 | 78 | 0.281 |

p = 0.06

### Example 9

### Results

### JAK3 Variant rs2302600 Association with OmpC (Positive/Negative)

### Table 9

TABLE 9

| | rs2302600 | | |
|---|---|---|---|
| OMPC_P(OMPC_P) | AA | CA | CC |
| Positive | 52 | 36 | 13 |
| | 51.49 | 35.64 | 12.87 |
| negative | 78 | 52 | 13 |
| | 54.55 | 36.36 | 9.09 |
| Statistic | DF | Value | Prob |
| Mantel-Haenszel | 1 | 0.6027 | 0.4375 |
| Chi-Square | | | |

### Example 10

### Results

### JAK3 Variant rs2302600 Association with Cbir (Positive/Negative)

### Table 10

TABLE 10

| | rs2302600 | | |
|---|---|---|---|
| cbir_p | AA | CA | CC |
| Positive | 76 | 51 | 16 |
| | 53.15 | 35.66 | 11.19 |
| negative | 52 | 36 | 10 |
| | 53.06 | 36.73 | 10.2 |
| Statistic | DF | Value | Prob |
| Mantel-Haenszel | 1 | 0.0102 | 0.9196 |
| Chi-Square | | | |

### Example 11

### Results

### JAK3 Variant rs2302600 Association with ASCA (Positive/Negative)

### Table 11

TABLE 11

| | rs2302600 | | |
|---|---|---|---|
| ASCA | AA | CA | CC |
| Positive | 76 | 62 | 18 |
| | 48.72 | 39.74 | 11.54 |
| negative | 55 | 27 | 9 |
| | 60.44 | 29.67 | 9.89 |
| Statistic | DF | Value | Prob |
| Mantel-Haenszel | 1 | 2.2129 | 0.1369 |
| Chi-Square | | | |

### Example 12

### Results

### JAK3 Variant rs2302600 Association with OmpC in Dominant Genetic Model

### Table 12

TABLE 12

| | rs2302600_dom | | |
|---|---|---|---|
| OMPC_P(OMPC_P) | 0 | 1 | Total |
| Positive | 52 | 49 | 101 |
| | 51.49 | 48.51 | |
| negative | 78 | 65 | 143 |
| | 54.55 | 45.45 | |
| Statistic | DF | Value | Prob |
| Chi-Square | 1 | 0.2227 | 0.637 |

### Example 13

### Results

### JAK3 Variant rs2302600 Association with Cbir in Dominant Genetic Model

### Table 13

TABLE 13

| | rs2302600_dom | | |
|---|---|---|---|
| cbir_p | 0 | 1 | Total |
| Positive | 76 | 67 | 143 |
| | 53.15 | 46.85 | |
| negative | 52 | 46 | 98 |
| | 53.06 | 46.94 | |
| Statistic | DF | Value | Prob |
| Mantel-Haenszel | 1 | 0.0002 | 0.9896 |
| Chi-Square | | | |

### Example 14

### Results

### JAK3 Variant rs3212741 Association with OmpC (Positive/Negative)

### Table 14

TABLE 14

| | rs3212741 | | |
|---|---|---|---|
| OMPC_P(OMPC_P) | CC | TC | TT |
| Positive | 73 | 27 | 1 |
| | 72.28 | 26.73 | 0.99 |
| negative | 93 | 45 | 3 |
| | 65.96 | 31.91 | 2.13 |
| Statistic | DF | Value | Prob |
| Mantel-Haenszel | 1 | 1.2813 | 0.2577 |
| Chi-Square | | | |

### Example 15

### Results

### JAK3 Variant rs3212741 Association with Anti-I2 (Positive/Negative)

### Table 15

TABLE 15

| | rs3212741 | | |
|---|---|---|---|
| I2_P(I2_P) | CC | TC | TT |
| Positive | 111 | 44 | 4 |
| | 69.81 | 27.67 | 2.52 |
| negative | 55 | 28 | 0 |
| | 66.27 | 33.73 | 0 |
| Statistic | DF | Value | Prob |
| Mantel-Haenszel | 1 | 0.0227 | 0.8803 |
| Chi-Square | | | |

### Example 16

### Results

### JAK3 Variant rs3212741 Association with Anti-Cbir (Positive/Negative)

### Table 16

TABLE 16

| | rs3212741 | | |
|---|---|---|---|
| cbir_p | CC | TC | TT |
| Positive | 104 | 36 | 2 |
| | 73.24 | 25.35 | 1.41 |
| negative | 60 | 35 | 2 |
| | 61.86 | 36.08 | 2.06 |
| Statistic | DF | Value | Prob |
| Mantel-Haenszel | 1 | 3.2641 | 0.0708 |
| Chi-Square | | | |

### Example 17

### Results

### JAK3 Variant rs3212741 Association with Anti-OmpC in Dominant Genetic Model

### Table 17

TABLE 17

| | rs3212741_dom | | |
|---|---|---|---|
| OMPC_P(OMPC_P) | 0 | 1 | Total |
| Positive | 73 | 28 | 101 |
| | 72.28 | 27.72 | |
| negative | 93 | 48 | 141 |
| | 65.96 | 34.04 | |
| Statistic | DF | Value | Prob |
| Chi-Square | 1 | 1.091 | 0.2962 |

### Example 18

### Results

### JAK3 Variant rs3212741 Association with Anti-I2 in Dominant Genetic Model

### Table 18

TABLE 18

rs3212741_dom

| I2_P(I2_P) | 0 | 1 | Total |
|---|---|---|---|
| Positive | 111 | 48 | 159 |
| | 69.81 | 30.19 | |
| negative | 55 | 28 | 83 |
| | 66.27 | 33.73 | |
| Statistic | DF | Value | Prob |
| Chi-Square | 1 | 0.3184 | 0.5726 |

### Example 19

### Results

### JAK3 Variant rs3212741 Association with Anti-Cbir in Dominant Genetic Model

### Table 19

TABLE 19

| | rs3212741_dom | | |
|---|---|---|---|
| cbir_p | 0 | 1 | Total |
| Positive | 104 | 38 | 142 |
| | 73.24 | 26.76 | |
| negative | 60 | 37 | 97 |
| | 61.86 | 38.14 | |
| Statistic | DF | Value | Prob |
| Chi-Square | 1 | 3.4684 | 0.0626 |

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

1. A method of diagnosing susceptibility to a subtype of Crohn's disease in an individual, comprising:
determining the presence or absence of one or more risk variants at the Janus kinases 3 (JAK3) genetic locus in the individual; and
determining the presence or absence of a positive expression of ASCA and/or anti-I2;
wherein the presence of one or more risk variants at the JAK3 locus and the presence of ASCA and/or anti-I2 expression is indicative of susceptibility in the individual to the subtype of Crohn's Disease.

2. The method of claim 1, wherein one of the one or more risk variants at the JAK3 locus comprises SEQ. ID. NO.: 1.

3. The method of claim 1, wherein one of the one or more risk variants at the JAK3 locus comprises SEQ. ID. NO.: 2.

4. The method of claim 1, wherein positive expression of ASCA and/or anti-I2 comprises a high level of expression relative to a healthy subject.

5. A method of diagnosing a subtype of Crohn's disease in an individual, comprising:
obtaining a sample from the individual;
assaying the sample for the presence or absence of a risk variant at the Janus kinases 3 (JAK3) genetic locus in the individual; and
diagnosing the subtype of Crohn's disease based upon the presence of the risk variant at the JAK3 genetic locus.

6. The method of claim 5, wherein the risk variant comprises SEQ. ID. NO.: 1 and/or SEQ. ID. NO.: 2.

7. The method of claim 5, wherein the presence of the risk variant is associated with a positive expression of ASCA and/or anti-I2.

8. The method of claim 7, wherein the positive expression of ASCA and/or anti-I2 comprises a high level of expression relative to a healthy subject.
